(19) United States
(12) Patent Application Publication
(10) Pub. No.: US 2022/0304941 A1
(43) Pub. Date: Sep. 29, 2022

(54) COMPOSITION FOR PREVENTING OR TREATING ORAL DISEASES

(71) Applicant: TECOZYME INC, Seoul (KR)

(72) Inventors: Dong Ho CHOI, Seoul (KR); Yoon NAM, Seoul (KR)

(73) Assignee: TECOZYME INC, Seoul (KR)

(21) Appl. No.: 17/596,481

(22) PCT Filed: Jun. 11, 2020
(86) PCT No.: PCT/KR2020/007606

§ 371 Date: Dec. 10, 2021

(30) Foreign Application Priority Data
Jun. 18, 2019 (KR) 10-2019-0071928

(51) Int. Cl. A61K 31/047; A61P 31/04; A61Q 11/02; A61K 36/23; A61K 8/34; A61K 8/9789

(52) U.S. Cl. CPC A61K 31/047; A61P 31/04; A61Q 11/02; A61K 36/23; A61K 8/345; A61K 8/9789

(57) ABSTRACT

The present disclosure pertains to a composition for preventing or treating oral diseases. A composition for preventing or treating oral diseases according to an embodiment of the present disclosure can not only effectively prevent or treat tooth decay or periodontitis by inhibiting bacterial activity in the oral cavity but can also be used for a long period of time due to being non-toxic to human, and can be used in various formulations, such as oral tablets, toothpastes, gargles, and oral patches.

### FIELD OF THE INVENTION

The present disclosure relates to a composition for preventing or treating oral diseases.

### BACKGROUND OF THE INVENTION

Dental caries, also known as tooth decay, and gingival diseases, such as periodontitis, also known as gum disease, are diseases with a very high incidence, and which cause various clinical symptoms such as pain, masticatory dysfunction, destruction of periodontal tissue, bad breath, and cold teeth, and are a major factor in causing tooth loss.

A tooth cavity mainly occurs on the chewing side of tooth or at the proximal surface between teeth and teeth, and they destruct enamel surrounding dentin, and thereby, dentin and dentinal tubules are exposed, and dentin hypersensitivity occurs. Such dental caries is mainly generated in the age group of early childhood to adolescence, whose enamel is not sufficiently strengthened.

Gum disease (periodontitis) known as gingival disease occurs due to the damage of periodontal ligament that connects tooth and the alveolar bone. As the ecosystem of oral microorganisms is disrupted due to various causes, chronic inflammation of the underlying tissues occurs. The alveolar bone and periodontal ligament below the periodontal tissue are absorbed by local excessive concentration of active oxygen and inflammatory signaling substances. Due to the damage of alveolar bone and periodontal ligament, the support of the alveolar bone for the tooth is reduced, and tooth mobility is increased. Cement surrounding the tooth root is exposed, causing the tooth to feel a cold symptom. This periodontal disease mainly occurs in adults after 30 years of age, and the reality is that most thereof are insensitive to this disease.

It is known that such tooth cavities and gum diseases are mainly caused by oral pathogens existing in the oral cavity.

It is known that about 800 kinds of microorganisms are parasitic or symbiotic in the human oral cavity. Although this microbial flora is controlled by enzymes secreted together with saliva, the inside of the oral cavity, which is rich in nutrients and moisture, has good conditions for microorganisms to grow, so the number of microorganisms in plaque on the tongue or tartar on the surface of teeth can greatly increase.

As a method of suppressing the proliferation of such oral pathogens, therapy to remove plaque or tartar, a habitat of the bacteria, is known, and a therapy using an antibacterial agent that acts on the pathogen itself or an antibiotic with bactericidal and bacteriostatic actions is known.

However, it is difficult to use antibiotics for an extended period because they can cause systemic side effects to the body, such as allergies, and can cause resistant bacteria in the oral cavity or cause superinfection.

In addition, the antibacterial agents used in mouth fresheners have not yet clearly revealed their working effect on bacteria in the oral cavity. There is controversy about the therapeutic effect thereof on gingival disease. Also, when used for a long time, side effects on oral tissues such as human toxicity may occur.

### DESCRIPTION OF THE INVENTION

### Technical Problem

The present specification provides a composition for preventing or treating oral diseases that can not only effectively prevent or treat dental caries or periodontitis by inhibiting bacterial activity in the oral cavity but can also be used for a long period of time due to being non-toxic to humans, and can be used in various formulations, such as oral tablets, toothpastes, gargles, and oral patches.

### Tools to Solve the Problem

The present specification provides a composition for preventing or treating oral diseases, wherein the composition includes 1, 2, 3-butanetriol.

In this connection, the 1,2,3-butanetriol may include at least one selected from the group consisting of (2S, 3S) 1,2,3-butanetriol, (2S, 3R) 1,2,3-butanetriol, (2R, 3S) 1,2,3-butanetriol, and (2R, 3R) 1,2,3-butanetriol.

In addition, the composition for preventing or treating oral diseases may preferably include 1,2,3-butanetriol in a concentration of about 0.001 wt. % to about 0.250 wt. %, or about 0.001 wt. % to about 0.150 wt. %, or about 0.001 to about 0.100 wt. %.

According to an embodiment of the present disclosure, the composition for preventing or treating oral diseases may further include an Ajiwain (Trachyspermum ammi) extract in addition to the above-mentioned 1,2,3-butanetriol.

In this connection, the Ajiwain extract may be extracted using one or more solvents selected from the group consisting of water, methanol, ethanol, propanol, isopropanol, butanol, acetone, ether, ethyl acetate, methylene chloride, n-hexane, hydrochloric acid, acetic acid, formic acid, diethyl ether, and cyclohexane from Ajiwain.

In addition, it may be preferable that the Ajiwain extract is extracted and concentrated in a ratio of about 10:1 to about 500:1, or about 15:1 to about 100:1, or about 15:1 to 50:1. Simultaneously, the composition for preventing or treating oral diseases may include the Ajiwain extract extracted and concentrated in the above ratio at a concentration of about 0.1 wt. % to about 10 wt. %.

According to another embodiment of the invention, the composition for preventing or treating oral diseases may further include one or more additives selected from the group consisting of a water-soluble zinc salt, B vitamins, vitamin C, and vitamin E, in addition to 1,2,3-butanetriol and Ajiwain extract.

Preferably, the additive may be included at a concentration of about 0.001 wt. % to about 10 wt. %.

More preferably, the water-soluble zinc salt may be included at a concentration of about 0.001 wt. % to about 0.300 wt. %. For B vitamins, especially vitamin B12, it may be included at a concentration of about 0.0001 wt. % to about 0.01 wt. %, For vitamin C, it may be included at a concentration of about 0.11 wt. % to about 10 wt. %, and for vitamin E, it may be included at a concentration of about 0.01 wt. % to about 5 wt. %.

The composition for preventing or treating oral disease according to an embodiment of the present disclosure, due to the aforementioned composition, the composition may have antibacterial, bactericidal, or bacteriostatic activity against one or more types of bacteria selected from the group consisting of Aggregatibacter actinomycetemcomitans, Tannerella forsythia, Treponema denticola, Prevotella nigrescens, Eubacterium nodatum, Parvimonas micra, Eikenella corrodens, Campylobacter rectus, Fusobacterium nucleatum (ATCC 25586), Streptococcus sobrinus, Streptococcus salivarius (GTCO215), Streptococcus anginasus (FW73), Streptococcus mutans (JCM5705), Prevotella intermedia (ATCC 25611), and Porphyromonas gingivalis (W83, ATCC 33277 (F), FDC 381).

In addition, the composition for preventing or treating oral diseases may be in a form processed into one or more formulations selected from the group consisting of toothpastes, mouthwashes, oral tablets, gums, candies, oral spray, oral ointment, oral varnish, and oral patches.

The terms “first”, “second”, etc., may be used herein to describe various elements, these terms are only used to distinguish one element from another element.

In addition, terms used in the present specification are used to describe exemplary embodiments and are not intended to limit the scope of the present disclosure.

The terms of a singular form may include plural forms unless otherwise specified.

As used herein, the terms “comprise,” “include,” “have,” and the like are intended to specify the presence of stated feature, integer, step, element, or combination thereof, but do not preclude the presence or addition of one or more other features, integers, steps, elements, or combinations thereof.

Since the present disclosure may have various modifications and various forms, specific embodiments will be illustrated and described in detail below. However, this is not intended to limit the present disclosure to the specific disclosed form, and it should be understood to include all modifications, equivalents and substitutions included in the spirit and technical scope of the present disclosure.

Hereinafter, the present disclosure will be described in detail.

According to one aspect of the present disclosure, there is provided a composition for preventing or treating oral diseases, wherein the composition includes 1,2,3-butanetriol.

Commonly used mouthwashes and the like, while having an antibacterial effect on tooth decay, contain a fluorine-based compound that can make teeth hard by binding to a calcium component of the teeth; antibiotics of a penicillin class, an erythromycin class, or a tetracycline class; antibacterial agents such as chlorhexidine; an alcohol-based compound such as ethanol, and exert an antibacterial effect on bacteria in the oral cavity.

However, fluorine-based compounds are highly toxic, and when swallowed, side effects such as fluorosis and indigestion may occur. In the case of antibiotics, resistant bacteria may emerge, and chlorhexidine-based antibacterial agents are also highly toxic to the human body and may cause discoloration of teeth and oral tissues.

As described above, after repeated research to supplement the disadvantages of the oral formulations that have been commonly used in the present, the inventors of the present disclosure found that 1,2,3-butanetriol has an effect of inhibiting bacterial growth in the oral cavity, and hardly generates toxicity to the human body, and thus can be used for a long period of time, and can be used in various formulations, such as oral tablets, toothpaste, gargles, and oral patches, and then completed the present disclosure.

1,2,3-butanetriol may be represented by the following chemical formula.

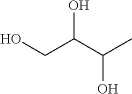

In other words, since 1,2,3-butanetriol has a form in which a hydroxy group is continuously bonded to 1,2, and 3 carbon positions of the butane (normal-butane) basic skeleton, a stereocenter is formed at the 2 and 3 carbon positions. Accordingly, it may have a total of four optical isomers of (2S, 3S) 1,2,3-butanetriol, (2S, 3R) 1,2,3-butanetriol, (2R, 3S) 1,2,3-butanetriol, and (2R, 3R) 1,2,3-butanetriol.

These four optical isomers do not appear to exhibit a significant difference between the respective compounds in the effect of inhibiting bacterial growth in the oral cavity of the human body. Accordingly, the composition for preventing or treating oral diseases according to an embodiment of the present disclosure may include at least one 1,2,3-butanetriol selected from the group consisting of (2S, 3S) 1,2,3-butanetriol, (2S, 3R) 1,2,3-butanetriol, (2R, 3S) 1,2,3-butanetriol, and (2R, 3R) 1,2,3-butanetriol, or all of them.

In this connection, the composition for preventing or treating oral diseases may include 1,2,3-butanetriol in an amount of about 0.001 wt. % to about 0.250 wt. %. The lower limit may be about 0.001 wt. % or more, or about 0.1 mM or more, and the upper limit may be about 25 mM or less, about 0.250 wt. % or less, about 0.150 wt. % or less, about 12.5 mM or less, about 0.100 wt. % or less, or about 10 mM or less.

When the concentration of 1,2,3-butanetriol is too low, the effect of inhibiting bacterial activity in the oral cavity may be reduced, which may not be unsuitable for preventing or treating oral diseases. When the concentration of 1,2,3-butanetriol is too high, due to toxicity to human cells, it may also be unsuitable for use in preventing or treating oral diseases.

In addition, according to an embodiment of the present disclosure, the composition for preventing or treating oral diseases may further include an Ajiwain (Trachyspermum ammi) extract in addition to the above-mentioned 1,2,3-butanetriol.

Ajiwain is an annual plant of the genus Ajiwain of the buttercup family, native to India, and its fruits are used as spices.

As used herein, the Ajiwain extract refers to an extract extracted from the seeds (fruit-like seeds) and leaves of the plants described above.

The inventors of the present disclosure have found that, when the above-mentioned 1,2,3-butanetriol and the Ajiwain extract are used together, the activity inhibiting effect on oral bacteria is greatly increased, as compared to the case of using each alone.

In this connection, the Ajiwain extract may be extracted using one or more solvents selected from the group consisting of water, methanol, ethanol, propanol, isopropanol, butanol, acetone, ether, ethyl acetate, methylene chloride, n-hexane, hydrochloric acid, acetic acid, formic acid, diethyl ether, and cyclohexane from Ajiwain.

In addition, it may be preferable that the Ajiwain extract is extracted and concentrated in a ratio of about 10:1 to about 500:1. Simultaneously, the composition for preventing or treating oral diseases may include the Ajiwain extract extracted and concentrated in the above ratio at a concentration of about 0.1 wt. % to about 10 wt. %.

When the content of the Ajiwain extract is too small, the synergistic effect is lost due to the mixed use with 1,2, 3-butanetriol.

In this regard, the extraction ratio may be preferably from about 15:1 to about 100:1, or from about 15:1 to 50:1, and the content of the Ajiwain extract may preferably be included at a concentration of from 0.01 wt. % to about 1 wt. %, or from about 0.02 wt. % to about 0.7 wt. %.

According to another embodiment of the invention, the composition for preventing or treating oral diseases may further include one or more additives selected from the group consisting of a water-soluble zinc salt, B vitamins, vitamin C, and vitamin E, in addition to 1,2,3-butanetriol and Ajiwain extract.

The water-soluble zinc salt is a salt of zinc (Zn), specifically, for example, zinc oxide, zinc chloride (ZnCl₂), zinc hydroxide (Zn(OH)₂), zinc sulfate (ZnSO₄), or zinc gluconate, and the like.

The water-soluble zinc salt may bind to an enzyme (catabolic enzyme) used for energy metabolism by anaerobic bacteria in the oral cavity, which is a causative bacterium of periodontal disease and bad breath, and inhibit enzyme activity, and may inhibit the action of MMP enzymes. For this reason, the composition for preventing or treating oral diseases containing a water-soluble zinc salt may have an antibacterial action that suppresses the survival and growth of anaerobic bacteria. In addition, it is possible to obtain a protective effect of suppressing the destruction of periodontal tissue.

B vitamins may be a complex including B1, B2, B3, B5, B6, B7, B9, and B12, and, among them, the composition for preventing or treating oral diseases according to an embodiment of the present disclosure may include B6 and B12.

These B vitamins can strengthen the immune system and nervous system functions of the human body, and promote metabolism, thereby helping heal wounds or inflammation in the oral cavity.

Vitamin C, a water-soluble vitamin, also called ascorbic acid, is a representative antioxidant, which is generated during the antibacterial process of neutrophils and macrophages concentrated in chronic inflammatory processes and can remove active oxygen that can destroy periodontal tissue. Thereby, the composition for preventing or treating oral diseases containing vitamin C can obtain a gum protective effect.

Vitamin E is a fat-soluble vitamin, including four tocopherols, including alpha-tocopherol, beta-tocopherol, gamma-tocopherol, and delta-tocopherol, and four tocotrienol components, including alpha-tocotrienol, beta-tocotrienol, gamma-tocotrienol, and delta-tocotrienol.

When the composition for preventing or treating oral diseases according to an embodiment of the present disclosure includes the above-mentioned vitamin E, it is generated during the antibacterial process of neutrophils and macrophages concentrated in the chronic inflammatory process and can destroy periodontal tissue and can remove active oxygen and also promote collagen synthesis, helping to regenerate damaged oral tissues.

In this connection, the additive may be included at a concentration of about 0.001 wt. % to about 10 wt. %.

Specifically, the water-soluble zinc salt may be included at a concentration of about 0.001 wt. % to about 0.300 wt. %. For B vitamins, especially vitamin B12, it may be included at a concentration of about 0.0001 wt. % to about 0.01 wt. %, For vitamin C, it may be included at a concentration of about 0.11 wt. % to about 10 wt. %, and for vitamin E, it may be included at a concentration of about 0.01 wt. % to about 5 wt. %.

The composition for preventing or treating oral disease according to an embodiment of the present disclosure, due to the aforementioned composition, the composition may have antibacterial, bactericidal, or bacteriostatic activity against one or more types of bacteria selected from the group consisting of Aggregatibacter actinomycetemcomitans, Tannerella forsythia, Treponema denticola, Prevotella nigrescens, Eubacterium nodatum, Parvimonas micra, Eikenella corrodens, Campylobacter rectus, Fusobacterium nucleatum (ATCC 25586), Streptococcus sobrinus, Streptococcus salivarius (GTCO215), Streptococcus anginasus (FW73), Streptococcus mutans (JCM5705), Prevotella intermedia (ATCC 25611), and Porphyromonas gingivalis (W83, ATCC 33277 (F), FDC 381).

Porphyromonas gingivalis (W83, ATCC 33277 (F), FDC 381) is known as a pathogen that mainly causes periodontitis, and Prevotella intermedia (ATCC 25611) is known as a pathogen that mainly causes gingivitis.

Fusobacterium nucleatum (ATCC 25586) is known as a common oral bacterium that is involved in vascular diseases in various gums or tooth decay. Recently, Fusobacterium nucleatum (ATCC 25586) is also known to promote cancer cell growth in colon cancer.

Streptococcus sobrinus, Streptococcus salivarius (GTCO215), Streptococcus anginasus (FW73) and Streptococcus mutans (JCM5705) are the representative bacteria that cause tooth decay. In the past, dental caries caused by Streptococcus mutans (JCM5705) was widely known. However, recently, Streptococcus sobrinus, Streptococcus salivarius (GTCO215), and Streptococcus anginasus (FW73) are known to play an important role in the progression of tooth decay. In particular, when two or more types of bacteria coexist in the oral cavity, it is known that the incidence rate of tooth decay is high, and the progression speed is also fast.

The composition for preventing or treating oral diseases according to an embodiment of the present disclosure has antibacterial, bactericidal, or bacteriostatic activity against the major pathogens of the above-described oral diseases.

In addition, due to this bacterial inhibition effect, it is possible to effectively prevent or treat gum-related diseases such as periodontitis, or tooth-related diseases such as tooth decay.

In addition, the composition for preventing or treating oral diseases may be in a form processed into one or more formulations selected from the group consisting of toothpaste, mouthwash, oral tablet, gum, candy, oral spray, oral ointment, oral varnish, and oral patches.

For example, when the composition for preventing or treating oral diseases of the present disclosure is a toothpaste formulation, it may include a fluorine compound, a wetting agent, an abrasive, a binder, a foaming agent, a flavoring agent, a sweetener, a colorant, a preservative, a solvent, a pH adjusting agent, and the like.

When the composition for preventing or treating oral diseases of the present disclosure is mouthwash, it may include water as a solvent, and may include a surfactant, an excipient, a colorant, a spice, a preservative, a stabilizer, and a pH adjusting agent, and the like commonly used to formulate active ingredients.

In addition, in the case of oral tablets, gums, candy, oral sprays, oral ointments, oral varnishes, and oral patches (films), the composition may have a form generally used in the technical field to which the present disclosure pertains. In addition, depending on each formulation, the composition may also include additives commonly used for the preparation of the corresponding formulation in the technical field to which the present disclosure pertains.

### Advantage of the Invention

The composition for preventing or treating oral diseases of the present disclosure can not only effectively prevent or treat tooth decay or periodontitis by inhibiting the activity of various types of bacteria present in the oral cavity, among them, pathogens that mainly cause oral diseases, but can also be used for a long period of time due to being non-toxic to human, and can be used in various formulations, such as oral tablets, toothpastes, gargles, and oral patches.

### BRIEF DESCRIPTION OF THE DRAWINGS

FIG. 1 is a graph illustrating the culture viability of human fibroblasts according to the concentration change of 1, 2,3-butanetriol.

FIGS. 2 and 3 are graphs illustrating the activity inhibitory effect of 1,2,3-butanetriol on oral bacteria.

FIG. 4 is a graph illustrating the activity inhibitory effect of 1,2,3-butanetriol and Ajiwain extract on oral bacteria.

### DETAILED DESCRIPTION OF THE INVENTION

Hereinafter, functions and effects of the present disclosure will be described in detail by specific examples of the present disclosure. Meanwhile, the examples are provided only to illustrate the present disclosure, and the scope of the invention is not limited thereto.

### Examples

1,2,3-butanetriol was used by purchasing a commercially available racemic mixture of optical isomers. (product of Sigma-Aldrich)

The Ajiwain extract was used as an extract obtained by extracting the seeds and leaves of Ajiwain (Trachyspermum ammi) in a ratio of about 20:1 using water.

Cytotoxicity Test

First, to examine the biotoxicity of 1,2,3-butanetriol, culture survival experiments of human fibroblasts according to the concentration change of 1,2,3-butanetriol were conducted.

The initial seeding density was 5000 cells/well based on 96 wells, and the culture conditions were as follows:

Dulbecco's Modified Eagle Medium (DMEM)+Fetal Bovine Serum (FBS) 10%; 37° C., 5% CO₂; and 12 hours after seeding, 1,2,3-butanetriol was treated at different concentrations to determine cytotoxicity, and after 48 hours of 1,2,3-butanetriol treatment, Dojindo Cell Counting Kit-8 was dispensed at a rate of 10 μl per well and incubated at 37° C. for 2 hours, then cell viability was measured at 450 nm Absorbance.

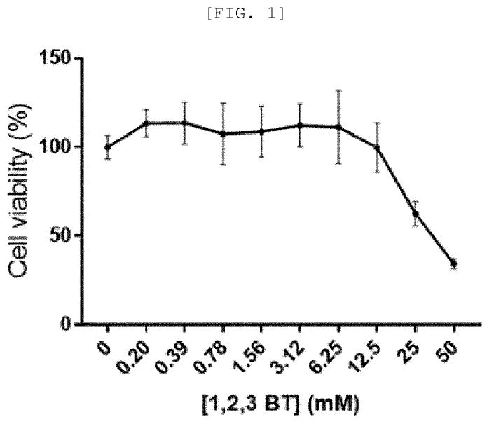

The measurement results are shown in FIG. 1.

FIG. 1 is a graph illustrating the culture viability of human fibroblasts according to the concentration change of 1, 2,3-butanetriol, and the horizontal axis of FIG. 1 indicates the concentration of the treated 1,2,3-butanetriol in an mM unit.

Referring to FIG. 1, it was identified that the cell viability as not significantly reduced at a concentration of about 12.5 mM or less, and the cell viability is about 50% at a concentration of about 25 mM.

Antibacterial test: 1,2,3-butanetriol

Subsequently, to examine the effect of inhibiting bacterial activity in plaque, bacterial strains were prepared as follows.

First, plaque was collected from three research participants, and was cultured in a BHI medium (Bovine Heart Infusion 4 g/160 ml distilled water-DDW) with 1 wt. % sucrose added for 24 hours.

From the above cultured strain, gDNA was extracted, and the bacteria were identified through PCR.

The identified bacterial species are shown in Table 1 below.

TABLE 1

| Bacterial species | Abbreviations |
|---|---|
| Aggregatibacter actinomycetemcomitans | Aa |
| Porphyromonas gingivalis | Pg |
| Tannerella forsythia | Tf |
| Treponema denticola | Td |
| Fusobacterium nucleatum | Fn |
| Prevotella nigrescens | Pn |
| Streptococcus mutans | Sm |
| Prevotella intermedia | Pi |
| Eubacterium nodatum | En |
| Parvimonas micra | Pm |
| Eikenella corrodens | Ec |
| Total bacterial load | Tb |
| Campylobacter rectus | Cr |
| Streptococcus sobrinus | Ss |

BHI medium (Bovine Heart Infusion 4 g/160 ml distilled water-DDW) with 1wt. % sucrose added was prepared and sterilized in a high-temperature and high-pressure autoclave. Then, 1,2, 3-butanetriol (1,2,3-BT) was dispensed to each concentration, and a 96-well microplate containing 180 μl of the culture solution was prepared.

Here, 20 μl of the culture solution in which the bacteria in plaque prepared above was proliferated was dispensed per well.

This microplate was placed in a Biotek Synergy 2 microplate reader, the temperature was adjusted to 37° C., and the absorption rate was measured at 600 nm.

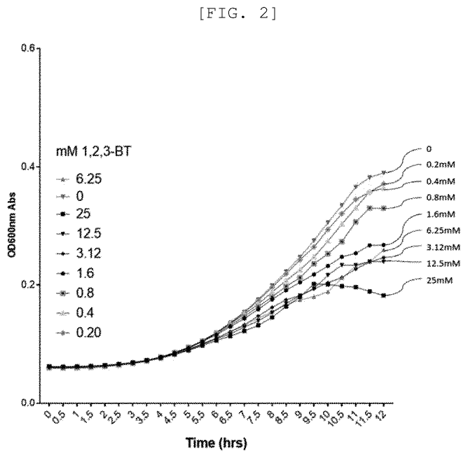

The measurement results are shown in FIG. 2.

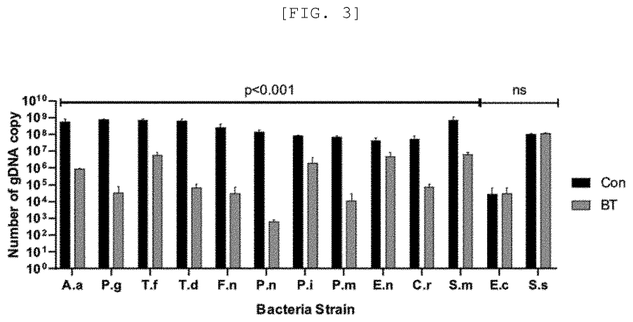

Separately from the absorption rate measurement, gDNA was extracted from the well microplate after treatment with butanetriol, and the results identified by PCR are shown in FIG. 3.

FIGS. 2 and 3 are graphs illustrating the activity inhibitory effect of 1,2,3-butanetriol on oral bacteria.

Referring to FIG. 2, it was identified that when 1,2, 3-butanetriol is treated, bacterial growth was significantly effectively inhibited. It was clearly identified that when 12 hours have elapsed after culturing, an effect of inhibiting bacterial growth of at least about 10% or more, and a maximum of about 50% or more was exhibited, compared to the case in which the 1,2,3-butanetriol is not treated. Referring to FIG. 3, the growth inhibitory effect of 1,2,3-butanetriol on each bacterial species was clearly identified.

Antibacterial test: 1,2,3-butanetriol+Ajiwain extract

To identify the synergistic effect produced when an Ajiwain extract is used, BHI medium (Bovine Heart Infusion 4 g/160 ml distilled water-DDW) with 1 wt. % sucrose added was prepared and sterilized. Then, 1,2,3-butanetriol (1,2,3-BT) and Ajiwain extract were dispensed to each concentration, and a 96-well microplate containing 180 μl of the culture solution was prepared.

1,2,3-Butanetriol concentration: 0.66 mg/ml

Ajiwain extract concentration: AZ0.05:0.05 mg/ml; AZ0.5:0.5 mg/ml

Here, 20 μl of the culture solution in which the bacteria in plaque prepared above was proliferated was dispensed per well.

This microplate was placed in a Biotek Synergy 2 microplate reader, the temperature was adjusted to 37° C., and the absorption rate was measured at 600 nm.

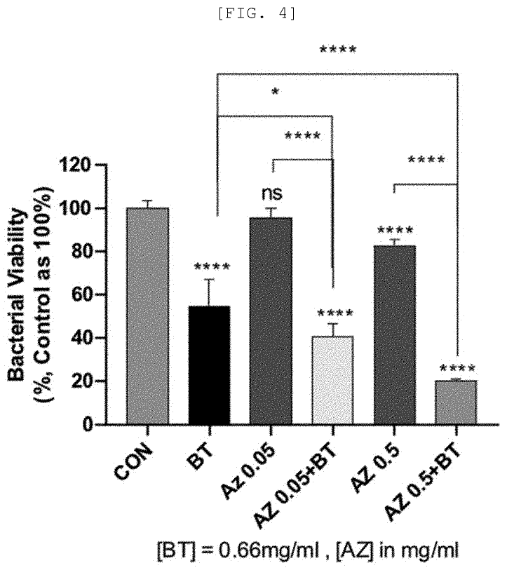

The measurement results are shown in FIG. 4.

Referring to FIG. 4, compared to the case where only 1,2,3-butanetriol was treated alone, when 1,2,3-butanetriol and Ajiwain extract were simultaneously treated, it was clearly identified that the effect of inhibiting bacterial growth was greatly increased. When the Ajiwain extract was used in high concentration (AZ50+BT), it was identified that it exhibited about 80% effect of inhibiting bacterial growth compared to the case without any treatment (CON).

1. A composition for preventing or treating oral diseases, wherein the composition includes 1,2,3-butanetriol.

2. The composition according to claim 1, wherein the 1,2,3-butanetriol includes at least one selected from the group consisting of (2S, 3S) 1,2,3-butanetriol, (2S, 3R) 1,2,3-butanetriol, (2R, 3S) 1,2,3-butanetriol, and (2R, 3R) 1,2,3-butanetriol.

3. The composition according to claim 1, wherein the 1,2,3-butanetriol is included in a concentration of 0.001 wt. % to 0.250 wt. %.

4. The composition according to claim 1, further including an Ajiwain (Trachyspermum ammi) extract.

5. The composition according to claim 4, wherein the Ajiwain extract is extracted using one or more solvents selected from the group consisting of water, methanol, ethanol, propanol, isopropanol, butanol, acetone, ether, ethyl acetate, methylene chloride, n-hexane, hydrochloric acid, acetic acid, formic acid, diethyl ether, and cyclohexane from Ajiwain.

6. The composition according to claim 4, wherein the Ajiwain extract is extracted and concentrated in a ratio of 10:1 to 500:1, and wherein the Ajiwain extract is included at a concentration of 0.1 wt. % to 1 wt. %.

7. The composition according to claim 1, further including one or more additives selected from the group consisting of a water-soluble zinc salt, B vitamins, vitamin C, and vitamin E.

8. The composition according to claim 7, wherein the additive is included in a concentration of 0.001 wt. % to 10 wt. %.

9. The composition according to claim 1, wherein the composition has antibacterial activity against one or more types of bacteria selected from the group consisting of Aggregatibacter actinomycetemcomitans, Tannerella forsythia, Treponema denticola, Prevotella nigrescens, Eubacterium nodatum, Parvimonas micra, Eikenella corrodens, Campylobacter rectus, Fusobacterium nucleatum (ATCC 25586), Streptococcus sobrinus, Streptococcus salivarius (GTCO215), Streptococcus anginasus (FW73), Streptococcus mutans (JCM5705), Prevotella intermedia (ATCC 25611), and Porphyromonas gingivalis (W83, ATCC 33277 (F), FDC 381).

10. The composition according to claim 1, wherein the composition has one or more formulations selected from the group consisting of toothpastes, mouthwashes, oral tablets, gums, candies, oral spray, oral ointment, oral varnish, and oral patches.

11. A method for preventing or treating oral diseases comprising:
administering a composition of claim 1.

12. The method according to claim 11, wherein the 1,2,3-butanetriol is selected from the group consisting of (2S, 3S) 1,2,3-butanetriol, (2S, 3R) 1,2,3-butanetriol, (2R, 3S) 1,2,3-butanetriol, and (2R, 3R) 1,2,3-butanetriol.

13. The method according to claim 11, wherein the 1,2,3-butanetriol is included in a concentration of 0.001 wt. % to 0.250 wt. % of the total composition.

14. The method according to claim 11, further including an Ajiwain (Trachyspermum ammi) extract.

15. The method according to claim 14, wherein the Ajiwain extract is extracted using one or more solvents selected from the group consisting of water, methanol, ethanol, propanol, isopropanol, butanol, acetone, ether, ethyl acetate, methylene chloride, n-hexane, hydrochloric acid, acetic acid, formic acid, diethyl ether, and cyclohexane from Ajiwain.

16. The method according to claim 14, wherein the Ajiwain extract is extracted and concentrated in a ratio of 10:1 to 500:1, and wherein the Ajiwain extract is included at a concentration of 0.1 wt. % to 1 wt. %.

17. The method according to claim 11, further including one or more additives selected from the group consisting of a water-soluble zinc salt, B vitamins, vitamin C, and vitamin E.

18. The method according to claim 17, wherein the additive is included in a concentration of 0.001 wt. % to 10 wt. %.

19. The method according to claim 11, wherein the composition has antibacterial activity against one or more types of bacteria selected from the group consisting of Aggregatibacter actinomycetemcomitans, Tannerella forsythia, Treponema denticola, Prevotella nigrescens, Eubacterium nodatum, Parvimonas micra, Eikenella corrodens, Campylobacter rectus, Fusobacterium nucleatum (ATCC 25586), Streptococcus sobrinus, Streptococcus salivarius (GTCO215), Streptococcus anginasus (FW73), Streptococcus mutans (JCM5705), Prevotella intermedia (ATCC 25611), and Porphyromonas gingivalis (W83, ATCC 33277 (F), FDC 381).

20. The method according to claim 11, wherein the composition has one or more formulations selected from the group consisting of toothpastes, mouthwashes, oral tablets, gums, candies, oral spray, oral ointment, oral varnish, and oral patches.
